(19) United States
(12) Patent Application Publication
(10) Pub. No.: US 2020/0054454 A1
(43) Pub. Date: Feb. 20, 2020

(54) BONE RECONSTRUCTION IMPLANT

(71) Applicant: Paul S. D'URSO, Richmond (AU)

(72) Inventors: Paul S. D'URSO, Richmond (AU); Philip LEWIS, Richmond (AU)

(21) Appl. No.: 16/344,887

(22) PCT Filed: Oct. 31, 2017
(86) PCT No.: PCT/AU2017/051195

§ 371 Date: Apr. 25, 2019

(30) Foreign Application Priority Data
Oct. 31, 2016 (AU) 2016904443

(51) Int. Cl. A61F 2/28; A61B 5/01; A61B 5/145; A61B 8/08; A61N 1/05; A61N 2/02; A61B 5/11; A61B 5/0478

(52) U.S. Cl. CPC A61F 2/2875; A61B 5/01; A61B 5/145; A61B 5/0478; A61N 1/0539; A61N 2/02; A61B 5/1112; A61B 8/0808

(57) ABSTRACT

Bone reconstruction implants and systems comprising the implants are provided. The implants and systems find use in monitoring and/or modulating the health of a patient. The bone reconstruction implants may be contoured to conform to the surface contours of a bony defect and comprise one or more functional elements. The functional elements may be positioned within the bone reconstruction implant so as to maximize alignment or interaction with functional elements in implanted medical devices, internal anatomical features or external devices.

### FIELD

The present disclosure relates to bone reconstruction implants and to systems and methods which include the implants. The disclosure particularly relates to patient specific bone reconstruction implants comprising one or more functional elements. The implants, systems and methods find use in the delivery of healthcare.

### BACKGROUND

Craniotomy and craniectomy are two procedures in which an area of skull bone is removed from a patient. In the case of craniotomy, the skull bone is replaced at the end of the surgical procedure, whereas in a craniectomy, the skull bone is left out for a period of time, as determined by the treating physician and the condition of the patient.

Craniectomy is performed for a variety of reasons, including, for example, facilitating evacuation of collected intracranial fluid, removal of tumours, removal of irrepairably damaged or contaminated skull bone, or to allow the brain to swell without causing dangerous increases in pressure.

Craniotomy may be indicated for the same conditions as craniectomy, however additional reasons for performing a craniotomy include, for example, providing access to the brain for implanting medical devices. Examples of such devices include sensors, fluid/pressure control devices, devices intended to modulate brain function and any associated components or devices on which the operation of the aforementioned devices depend.

In cases where the bone is not replaced with the original bone flap that is removed, an implant must be placed to permit restoration of skull anatomy. A variety of materials can be used to fashion the implant, for example, metal, plastic/polymer or ceramic. The implant is typically constructed by hand to anatomically match the defect in the skull, however the implant may also be constructed using additive manufacturing techniques or traditional metal/polymer forming techniques such as casting, blow-moulding and so on. The surgical procedure performed to implant the bone replacement is called a cranioplasty.

After a cranioplasty or craniotomy has been performed, there is often a need for the continued use of diagnostic or therapeutic technology, or both, in the ongoing care or rehabilitation of the patient. This technology is typically implanted deep into the brain substance (for example into deep white matter or the basal ganglia), a lesser distance into the brain substance (for example into the cortex or subcortical white matter), into the ventricles, onto the brain surface, outside the dura or onto the skin.

Such technology may be connected to or integrated with supporting technology that itself may be implanted. For example, in the case of deep brain stimulators, a small casing containing the battery and electronics required to drive stimulating electrodes is often implanted subcutaneously on the chest, connected by a wire. An alternative example is illustrated by the case of subdural electrodes, which rest on the brain surface and may be connected to an extracranial, subcutaneously-implanted casing similar to that used with deep brain stimulators, or indeed the casing may be integrated into the subdural electrode array itself and thus also be implanted intracranially.

In other situations, the additional technology may be external to the body. This may be because the technology is too large to be implanted, such as in the case of a microdialysate analyser, or that the state of technology development at the time is such that no commercially-available, implantable version of the device exists. An illustrative case is given by intracranial pressure monitoring technology, which has been in use clinically for over 30 years, however a commercially-available, fully-implantable and wirelessly-operated intracranial pressure monitor has only recently been realised. A further reason for the use of intracranially-implanted technology connected to external supporting technology may be that the clinical need is transient, and thus the implant is temporary.

In cases where the supporting technology is external to the body, connection to the implantable technology may be facilitated by a percutaneous lead. As such, the implanted device may only be used for a limited period of time, owing to the risk of infection and leakage of cerebrospinal fluid brought about by the need for a hole through the skin, skull and dura permitting access to the brain. Therefore, a risk-benefit decision must be made in relation to the period of time over which the implant may remain in-situ, weighing the ongoing risk of infection due to the presence of a hole in the skin, skull and dura against the clinical benefit to the patient. As a result, while some patients may benefit from longer-term monitoring or therapy achieved using an implantable device, the length of its application is limited.

U.S. Pat. No. 7,917,222 describes a cerebral and/or interface system with a housing mechanism configured to be at least partially located in a cavity formed in the subject's skull. The interface system is described as a system “having at least one device that is configured to operatively sense, monitor, store, record, log, transmit, analyze, quantify, detect changes in, predict changes in, warn of changes in, modify, or control the state or activity in at least one organ”. The patent describes that the housing mechanism for the interface system is configured to be situated at least in part within a bone of a subject. However, the bone of a subject is not a suitable carrier, substrate or housing for interface devices that are required to simply be implanted, or are required to remain in close proximity to a target structure that is ordinarily in close proximity with the bone. An example of a case in which the bone is not a suitable substrate, carrier or housing for an interface device is after a decompressive craniectomy, wherein the bone may be purposefully removed, and in certain circumstances, is purposefully disposed of due to irreversible contamination or damage. Other circumstances include cancer of the bone and infection of the bone.

U.S. Pat. No. 8,454,701 describes surgical methods and a device for fixing a housing device to a receiving bone, into which an interface device may be inserted. However, the disclosure does not account for possible variations in the shape and size and complexities therein of the opening in the receiving bone that may themselves be dictated by the clinical needs of the patient, rather than the shape of the interface device and/or interface housing, and thus be unpredictable.

Clearly, the above described developments are not limited to application to a human skull and may find application into or onto any part of the body, be it human or animal.

Despite the above advances in the art it would be desirable to provide improved bone reconstruction implants, and systems and methods comprising such implants.

The reference in this specification to any prior publication (or information derived from it), or to any matter which is known, is not, and should not be taken as an acknowledgement or admission or any form of suggestion that that prior publication (or information derived from it) or known matter forms part of the common general knowledge in the field of endeavour to which this specification relates.

### SUMMARY

In one aspect of the present disclosure there is provided a bone reconstruction implant, said implant comprising one or more functional elements. The functional elements may be permanent, temporary or a combination of both permanent and temporary.

In another aspect there is provided a bone reconstruction implant, said implant comprising one or more functional elements, said implant being contoured in one or more surface regions, said contouring being based on patient specific computer imaging data, so that said one or more surface regions match or substantially match one or more surface contours of a bony defect in said patient.

Throughout this specification the term ‘substantially match’, in relation to matching of contoured surfaces, may mean that the respective surfaces have a hand and glove relationship, or a substantial hand and glove relationship.

In another aspect there is provided a bone reconstruction implant, said implant comprising one or more functional elements, said implant being contoured in one or more surface regions, said contouring being based on patient specific computer imaging data, so that said one or more surface regions match or substantially match one or more surface contours of a patient's internal anatomy.

In another aspect there is provided a bone reconstruction implant, said implant comprising one or more functional elements, said implant being contoured in one or more first surface regions, said contouring being based on patient specific computer imaging data, so that said one or more first surface regions match or substantially match one or more surface contours of a bony defect in said patient, said implant also being contoured in one or more second surface regions said contouring being based on patient specific computer imaging data, so that said one or more second surface regions match or substantially match one or more surface contours of a patient's internal anatomy.

In any of the herein disclosed aspects at least one of said functional elements in said bone reconstruction implant may be positioned within said implant such that, in use, the at least one functional element aligns with an internal anatomical feature of a patient.

By aligns it is meant that the position of the at least one functional element in the implant is such that its interaction with a target internal feature of the patient is substantially maximized.

The bone reconstruction implants of the present disclosure may be constructed to correct bony defects introduced by the performing of a surgical procedure and/or to correct bony defects resulting from disease or injury. The bone reconstruction implants may also be constructed to replace an entire bone. The bone reconstruction implants may directly or indirectly facilitate the delivery of healthcare.

The bone reconstruction implants may be manufactured to replace all or part of a patient's native bone. The bone reconstruction implants may be utilised as a substrate, physical support, carrier, encasing layer or otherwise, so as to provide a device for diagnostic or therapeutic technology. The present disclosure provides bone reconstruction implants that may facilitate the delivery of therapy or the diagnosis of a medical condition. Moreover, the present disclosure provides systems and methods utilising bone reconstruction implants that perform more than structural/load-bearing, articulating or cosmetic functions.

An implant designed to correct a bony defect may have infinitely variable dimensions and complexity and thus may therefore be patient-specific. An implant designed to replace an entire bone may have infinitely variable dimensions and complexity and thus may be patient-specific.

Further, to manufacture a bone reconstruction implant to fit, for example, a cranial defect, with infinitely variable dimensions and complexity, techniques that permit the manufacture of patient-specific prostheses may be used. Such techniques include, but are not limited to, using imaging devices and 3-D modelling software to produce digital patient-specific anatomical models, additive manufacturing to produce physical patient-specific biomodels, patient-specific biomodels fashioned by hand, patient-specific implants, and additive manufacturing techniques to produce patient-specific implants.

The bone reconstruction implants as herein disclosed may comprise one or more voids such that one or more functional elements of known geometry and proportions may be housed in the void, regardless of the size and complexity of the defect in the receiving bone.

The one or more voids may span the full thickness of one dimension of the implant. In another embodiment the one or more voids may span only a partial thickness of one dimension of the implant.

The one or more voids may be located entirely within the outer perimeter of the implant. Alternatively the one or more voids may constitute part of the outer perimeter of the implant.

The bone reconstruction implants may be contoured in one or more surface regions, said contouring being based on patient specific computer imaging data, so that said one or more surface regions match or substantially match one or more surface contours of a bony defect. Alternatively, or additionally, the implants may be contoured in one or more surface regions, said contouring being based on patient specific computer imaging data, so that said one or more surface regions match or substantially match one or more contours of a patient's internal anatomy. Alternatively, or additionally, the implant may be contoured in one or more surface regions, said contouring being based on patient specific computer imaging data, so that said one or more surface regions match or substantially match one or more contours of a patient's external anatomy.

In another aspect there is provided a bone reconstruction implant said implant comprising one or more functional elements, said implant being contoured in one or more surface regions, said contouring being based on patient specific computer imaging data, so that said one or more surface regions match or substantially match one or more surface contours of a bony defect in said patient, wherein one or more of the functional elements in the bone reconstruction implant comprises one or more attachments connected to the functional elements, said attachments extending beyond the physical borders of the bone reconstruction implant.

The one or more attachments may comprise a wire, a lead, a cable, a sheet or a layer or combinations thereof.

The one or more attachments may comprise at least one surface which conforms to the internal surface anatomy of a patient.

The one or more attachments may comprise at least one functional element as herein described.

The bone reconstruction implants may find use in the monitoring and/or modulation of the health of a patient.

Examples of functional elements in relation to any of the herein disclosed aspects or embodiments include, but are not limited to, sensors, for example, temperature sensors, biochemical sensors, mechanical sensors, electrical sensors, ultrasonic sensors or optical sensors; control elements, for example, fluid/pressure control elements; elements which modulate the functioning of a tissue, for example, stimulating electrodes, stimulating electromagnets, light sources, ultrasonic emitters; power generation elements; elements which deliver or receive energy. Functional elements may also include any associated components on which the operation of the aforementioned elements depend, for example electronics, tubing, wires, support structures or encasing structures.

The functional elements may be of any three dimensional shape. Preferred three dimensional shapes include, for example, a sphere, a spheroid, a cube, a rectangular prism, a prism, a triangular prism, a cylinder, a cone, a pyramid, or combinations thereof.

The functional elements may provide means for one or more of sensing, communicating, actuating, delivering or receiving information, delivering or receiving energy, or generating energy.

The means for sensing may be one or more of chemical, electrical, physical, optical or magnetic.

The means for stimulating may be one or more of chemical, electrical, physical, optical or magnetic.

The means for actuating may be one or more of pressure, vacuum or physical deformation.

The means for energy generation may be, for example, based on physical movement of the patient hosting the implant or based on other movements.

The functional elements may provide means for monitoring and/or measuring patient specific parameters and/or non-patient specific parameters.

Patient specific parameters include, but are not limited to, temperature, pressure, electrophysiological changes or the concentration and/or nature of one or more chemical species. Non-patient specific parameters include, but are not limited to, temperature, pressure, light intensity, electromagnetic radiation, sound, or the concentration and/or nature of one or more chemical species.

The one or more functional elements may provide means for stimulating the patient.

The one or more functional elements may be in communication with one or more other implanted devices. The one or more functional elements may deliver electrical power to, for example, another implanted device.

In some embodiments the functional elements may sense and quantify environmental parameters, for example, sound, electromagnetic radiation, thermal radiation or gravitational forces.

In some embodiments the functional elements may comprise GPS and satellite communication systems.

In some embodiments the functional elements may allow remote monitoring and feedback.

The one or more functional elements may be in communication with each other. The one or more functional elements may form a communication network.

The one or more functional elements may comprise one or more electronic components.

The one or more electronic components may provide means for one or more of sensing, stimulating, communicating, actuating, delivering or receiving information, delivering or receiving energy, or generating energy.

The computer imaging data may be obtained from one or more imaging devices, that is, any device or devices either singly or in combination that can capture and represent, in digital form, the external and/or internal anatomy of the human body (the anatomical data). Examples of such devices include, but are not limited to, Computed Tomography, Magnetic Resonance Imaging, Ultrasound, one or more lasers, one or more digital cameras, and medical ultrasound.

Using the anatomical data, the presently disclosed implants may be designed to fit a bony defect by either a person skilled in the use of three dimensional design software, or by using a set of processes automated in software.

In some embodiments, the bone reconstruction implant is a cranial reconstruction implant manufactured to correct a defect in the cranium. The defect may be introduced by the performing of a decompressive craniectomy.

The bone-reconstruction implant may be shaped to fit the unique shape of the bony defect of the intended recipient, and in this case is a ‘patient specific’ implant.

For example a cranial reconstruction implant may be shaped to fit the unique shape of a cranial defect. Alternatively, or additionally, the cranial reconstruction implant may also be contoured in one or more surface regions, so that said one or more surface regions match one or more contours of a patient's internal tissues. The internal tissues may be brain tissue.

In one embodiment the implant may follow the internal tissue contours. This is advantageous as it allows the implant to remain in contact with the tissue surface.

The shape and dimensions of the implant may be determined using computer software via automated processes, with human input or by a combination of both, or it may be determined entirely by human input without the use of a computer. In the latter case, it may be determined by using the techniques of hand sculpting, forming or moulding.

The bone reconstruction implant may be manufactured using a variety of materials as appropriate for its intended location, and the need for the implant to provide a load-bearing function or a largely cosmetic function or both. It will be appreciated that some materials offer differing physical properties that may be advantageous depending on the clinical application. For example, the passage of ultrasound through the bone-replacing implant, which may be required for the delivery of therapy or the diagnosis of a medical condition, is strongly impeded not only by bone, but also metals and some polymers (for example, polymethylmethacrylate). Thus in some embodiments, the bone-reconstruction implant may be manufactured from materials that permit the improved passage of ultrasound relative to bone. However, an implant that is also required to perform a load-bearing function may be required to be made from a metal, thus precluding the passage of ultrasound. It will be appreciated that a variety of materials may be used to manufacture bone-reconstruction implants which include, but are not limited to, soft or hard plastics/polymers, or materials with a continuum of physical properties that would render these materials as being soft, hard, or anywhere in between, metals (such as titanium and other metals or metal alloys that are amenable to implantation), ceramics, or composites of these materials. Those skilled in the art will appreciate that other biocompatible materials may be used to fabricated bone-reconstruction implants, and the examples given should not be taken to be limiting.

The bone reconstruction implant may be designed and manufactured in such a way that it contains one or more functional elements. The one or more elements may be permanently housed or the one or more elements may be removable. The bone-replacing implant may contain only permanently-housed elements, only removable elements, or it may contain a combination of permanently-housed and removable elements.

In some embodiments there is provided a bone reconstruction implant wherein at least one surface of said implant is contoured to match the contours of at least one surface of a bony defect, wherein at least one other surface of said implant is contoured to match the contours of at least one surface of internal tissue and wherein the implant comprises one or more functional elements.

In some embodiments there is provided a cranial reconstruction implant wherein at least one surface of said implant is shaped to match the shape of at least one surface of a cranial defect, wherein at least one other surface of said implant is shaped to match the shape of at least one surface of a patient's internal anatomical features and wherein the implant comprises one or more functional elements.

Internal anatomical features of the patient include, but are not limited to, vascular features, physical features, functional features and electrical features or combinations thereof. Vascular features, such as vascular topography, may be determined by, for example, vascular mapping. Electrical features may be determined by, for example, electroencephalography. The internal anatomical feature may be brain tissue.

In one embodiment the implants may comprise more than one functional element positioned within different regions of the implant.

The positioning of the one or more functional elements in the implant may be determined using computer software via automated processes.

For example computer imaging data may be obtained from one or more imaging techniques, that is, any technique either singly or in combination that can capture and represent, for example, in digital form, the internal anatomy of the human body (the internal anatomical data) of the patient.

Examples of such techniques include x-ray radiographic, magnetic resonance imaging, medical ultrasonography, medical ultrasound, endoscopy, elastography, tactile imaging, thermography, medical photography, positron emission tomography (PET) and single photon emission computed tomography.

Measurement and recording techniques which are not primarily designed to produce images, such as electroencephalography (EEG), magnetoencephalography (MEG) and electrocardiography (ECG) may also be employed to determine the positioning of the functional elements in the implant.

The one or more functional elements may be in communication with one or more remote devices. The one or more functional elements may form a network comprising one or more remote devices. Remote devices include, but are not limited to, mobile communication devices such as phones, remote computer servers and the like.

The one or more functional elements may be physically connected to one or more functional elements in another internal device implanted in the patient.

The connection may be via suitable male and female connectors. The connection may be temporary or permanent.

The one or more functional elements may be in wireless communication with an internally implanted device.

In another aspect of the present disclosure there is provided a method of manufacturing a bone reconstruction implant, in whole or in part, comprising:

(a) determining the contours of a bony defect of a patient in a region of interest using computer imaging;

(b) designing the implant, so that one or more regions of the implant match or substantially match one or more contours of said patient's bony defect,

(c) manufacturing the implant.

The implant may be manufactured using additive manufacturing. Alternatively, the implant may be manufactured by hand sculpting, forming or moulding.

The bone reconstruction implant may be a cranial reconstruction implant.

In another aspect of the present disclosure there is provided a method of manufacturing a bone reconstruction implant, in whole or in part, comprising:

(a) planning, using surgical planning software, a resection margin that defines the extent of removal of bone from the contours of a bony defect of a patient

(b) fabricating a resection template for intraoperative use using additive manufacturing techniques or other techniques amenable to manufacturing patient-specific anatomical models,

(c) designing the implant, so that it fits into the planned resection; and;

(d) manufacturing the implant.

The implant may be manufactured using additive manufacturing.

Alternatively, the implant may be manufactured by hand sculpting, forming or moulding.

The bone reconstruction implant may be a cranial reconstruction implant.

The bone resection margin may be a cranial resection margin.

The stereotactic plan may comprise a pre-manufactured contour matched resection template and/or a computer navigation device.

Advantageously this allows the implant to fit into the planned resection allowing a one stage resection and bone reconstruction to occur.

In another aspect of the present disclosure there is provided a system for monitoring the health of a patient and/or modulating the health of a patient, said system comprising:
(a) at least one bone reconstruction implant comprising at least one functional element, said implant being implanted to correct a bony defect in said patient; and
(b) at least one other device internal or external to said patient's anatomy; said at least one other device comprising one or more functional elements;
   wherein at least one functional element in the bone reconstruction implant and at least one functional element in the at least one other device are in electronic communication.

In another aspect of the present disclosure there is provided a system for monitoring the health of a patient and/or modulating the health of a patient, said system comprising:
(a) at least one bone reconstruction implant comprising at least one functional element, said implant being implanted to correct a bony defect in said patient; and
(b) at least one other device internal or external to said patient's anatomy; said at least one other device comprising one or more functional elements;
   wherein at least one functional element in the bone reconstruction implant and at least one functional element in the at least one other device are in electronic communication; and
   wherein the at least one bone reconstruction implant is contoured in one or more surface regions, said contouring being based on patient specific computer imaging data, so that said one or more surface regions match one or more contours of said patient's bony defect.

The bone reconstruction implant may alternatively be implanted to replace an entire bone. In this embodiment the surface regions are those of a patient's existing bone.

The bone reconstruction implants may comprise one or more voids such that one or more functional elements of predictable or known geometry and proportions may be housed in the void, regardless of the size and complexity of the defect in the receiving bone.

The one or more voids may span the full thickness of one dimension of the implant. In another embodiment the one or more voids may span only a partial thickness of one dimension of the implant.

The one or more voids may be located entirely within the outer perimeter of the implant. Alternatively the one or more voids constitute part of the outer perimeter of the implant.

The operation of the at least one functional element in the bone reconstruction implant may depend wholly or in part on the operation of the functional elements in the external device and/or in the other internal device.

The at least one functional element independently in each occurrence may comprise electronic components. The at least one external device may comprise at least one functional element. The functional element may comprise electronic components.

The placement of the functional element in the external device may be such that there is alignment between said element and the functional elements in the bone reconstruction implant. This is advantageous as communication or energy transmission between the implant and external device may be improved.

The one or more functional elements in the bone reconstruction implant implant may provide means for one or more of sensing, stimulating, communicating, actuating, delivering or receiving information, delivering or receiving energy, or generating energy.

The means for sensing may be one or more of chemical, electrical, physical, optical, acoustic or magnetic.

The means for stimulating may be one or more of chemical, electrical, physical, optical, acoustic or magnetic.

The means for actuating may be one or more of pressure, vacuum or physical deformation.

The means for energy generation may be, for example, physical movement of the patient having the implant or other movements.

The one or more functional elements may monitor and/or measure patient specific parameters and/or non-patient specific parameters.

Patient specific parameters include, but are not limited to, temperature, pressure, electrophysiological changes and the concentration of one or more chemical species. Non-patient specific parameters include, but are not limited to, temperature, pressure, light intensity, electromagnetic radiation, sound, and one or more chemical species.

In some embodiments, the functional elements in the bone reconstruction implant, for example a neural recording or neuromodulation element, may be powered and communicate to external electronics via one or more interfaces based on electromagnetic, optical, ultrasonic/acoustic or electrical means.

Wireless powering and/or recharging may be performed using techniques such as electromagnetic induction, radio-wave energy harvesting, piezoelectric conversion of ultrasound, or the photoelectric conversion of light.

The functional element in the bone reconstruction implant may be powered by and communicate to electronics in the external device via one or more interfaces based on one or more of electromagnetic, optical, ultrasonic and electrical means.

In some embodiments the external device may capture ambient data, such as pressure, temperature, chemical data, sound, light and so forth and, based on one or more of such data, instruct the functional element in the bone reconstruction implant to, for example, stimulate, measure, adjust, modify, or feedback.

In some embodiments, based on ambient data measurements, the external device may instruct the functional element in the bone reconstruction implant via the one or more communication interfaces to release therapeutic materials, such as drugs.

In some embodiments, based on ambient data measurements, the external device may instruct the functional element in the bone reconstruction implant to modulate the activity of a patient's tissue, for example provide neural stimulation.

In another aspect of the present disclosure there is provided a system for monitoring the health of a patient and/or modulating the health of a patient, said system comprising:
(a) at least one bone reconstruction implant, implanted to correct a bony defect in said patient, said at least one implant comprising one or more functional elements; and
(b) at least one external medical device positioned on said patient's external anatomy, said external device comprising one or more functional elements;
   wherein the at least one bone reconstruction implant and at least one external device are in communication; and
   wherein the at least one bone reconstruction implant comprises one or more surfaces, said one or more surfaces having at least one region contoured to substantially match at least one contour of a bony defect.

In another aspect of the present disclosure there is provided a system for monitoring the health of a patient and/or modulating the health of a patient, said system comprising:
(a) at least one bone reconstruction implant, implanted to correct a bony defect in said patient, said at least one implant comprising one or more functional elements; and
(b) at least one external medical device positioned on said patient's external anatomy, said external device comprising one or more functional elements;
   wherein the at least one bone reconstruction implant and at least one external device are in communication; and
   wherein the at least one bone reconstruction implant comprises one or more surfaces, said one or more surfaces having at least one region contoured to substantially match at least one contour of a bony defect; and
   wherein the at least one external device comprises one or more surfaces, said one or more surfaces having at least one region contoured to substantially match at least one contour of an external anatomical surface of the patient.

The bone reconstruction implants may comprise one or more voids such that one or more functional elements of predictable or known geometry and proportions may be housed in the void, regardless of the size and complexity of the defect in the receiving bone.

The one or more voids may span the full thickness of one dimension of the implant. In another embodiment the one or more voids may span only a partial thickness of one dimension of the implant.

The one or more voids may be located entirely within the outer perimeter of the implant. Alternatively the one or more voids constitute part of the outer perimeter of the implant.

The one or more contoured surface regions of the external medical device may comprise a part or a whole of any one or more of the surfaces of the device that are, in use, in contact with the external anatomical surface of the wearer.

The one or more contoured surface regions of the external medical device may be designed so that the device closely fits an external anatomical surface of the wearer.

By ‘substantially match’ it may mean that the contoured surface region of the external medical device fits an external anatomical surface of a wearer without kinks or overlaps.

The one or more contoured surface regions of the external medical device may be determined by wearer specific computer imaging data or other processes.

The shape and dimensions of the one or more contoured surface regions of the external medical device may be determined using computer software via automated processes, or via human input or by a combination of both

The shape and dimensions of the one or more contoured surface regions of the external medical device may be determined entirely by human input without the use of a computer.

The shape and dimensions of the one or more contoured surface regions of the external medical device may be determined by using the techniques of, for example, hand crafting or sculpting, forming or moulding.

In one embodiment the one or more contoured surface regions of the external medical device may substantially match the contours of a specific region of the external anatomical surface of the wearer. For example, to substantially match the external anatomical surface of all or part of the head, neck, chest, abdomen, pelvis or limbs of the wearer.

In one embodiment the one or more contoured surface regions of the external medical device may substantially match the contours of more than one region of the external anatomical surface of the wearer. For example, the external medical device may be a body suit or any part thereof.

The external medical device may be a full body suit, a shirt, a vest, a head band or a head cap.

The one or more functional elements may be positioned and/or orientated within the external medical device so that, in use, they substantially align with specific regions of the external anatomical surface of the wearer.

Examples of functional elements include, but are not limited to, sensors, for example, temperature sensors, biochemical sensors, mechanical sensors, electrical sensors, ultrasonic sensors or optical sensors; control elements, for example, fluid/pressure control elements; elements which modulate the functioning of a tissue, for example, stimulating electrodes, stimulating electromagnets, light sources, ultrasonic emitters; power generation elements; elements which deliver or receive energy. Functional elements may also include any associated components on which the operation of the aforementioned elements depend, for example electronics, tubing, wires, support structures or encasing structures.

The functional elements in the external medical device may be of any three dimensional shape. Preferred three dimensional shapes include, for example, a sphere, a spheroid, a cube, a rectangular prism, a prism, a triangular prism, a cylinder, a cone, a pyramid, or combinations thereof.

The functional elements may provide means for one or more of sensing, communicating, actuating, delivering or receiving information, delivering or receiving energy, or generating energy.

The means for sensing may be one or more of chemical, electrical, physical, optical or magnetic.

The means for stimulating may be one or more of chemical, electrical, physical, optical or magnetic.

The means for actuating may be one or more of pressure, vacuum or physical deformation.

The means for energy generation may be, for example, based on physical movement of the patient or based on other movements.

The functional elements may provide means for monitoring and/or measuring patient specific parameters and/or non-patient specific parameters.

Patient specific parameters include, but are not limited to, temperature, pressure, electrophysiological changes or the concentration and/or nature of one or more chemical species.

Non-patient specific parameters include, but are not limited to, temperature, pressure, light intensity, electromagnetic radiation, sound, or the concentration and/or nature of one or more chemical species.

The one or more functional elements may provide means for stimulating the wearer.

The one or more functional elements may be in communication with one or more other implanted devices. The one or more functional elements may deliver electrical power to, for example, an implanted device, including a bone reconstruction implant.

In one embodiment the functional elements may sense and quantify environmental parameters, for example, light, sound, electromagnetic radiation, thermal radiation or gravitational forces.

In one embodiment the functional elements may comprise GPS and satellite communication systems.

In one embodiment the functional elements may allow remote monitoring and feedback.

The one or more functional elements may be in communication with each other. The one or more functional elements may form a communication network.

The one or more functional elements may comprise one or more electronic components.

The one or more electronic components may provide means for one or more of sensing, stimulating, communicating, actuating, delivering or receiving information, delivering or receiving energy, or generating energy.

In one embodiment the external medical device may comprise more than one functional element positioned within different regions of the device. For example, the external medical device may be a body suit or parts thereof.

The shape and dimensions of the one or more contoured surface regions of the external medical device may be determined using computer software via automated processes.

For example computer imaging data may be obtained from one or more imaging devices, that is, any device or devices either singly or in combination that can capture and represent, in digital form, the external anatomy of the human body (the external anatomical data) of a patient. Examples of such devices include, but are not limited to, Computed Tomography, Magnetic Resonance Imaging, Ultrasound, one or more lasers, one or more digital cameras, and medical ultrasound.

When positioned on a patient's head the external device may be headgear. Examples of headgear include, but are not limited to, a helmet, a cap, and a headband.

Using the anatomical data, the external device may be designed to fit the intended patient by either a person skilled in the use of three dimensional design software, or by using a set of processes automated in software.

In one embodiment the external device may comprise a sheet of, for example, a thermoplastic material. The external device may comprise a thin sheet. The external device may comprise an impact-resistant thermoplastic. The external device may be aesthetically-pleasing.

The external device, for example sheet, may have a thickness between about 0.05 mm and 10 mm, or between about 0.1 mm and about 5 mm, or between about 0.2 mm and about 2 mm. The external device, for example sheet, may have a thickness less than 10 mm, or less than 5 mm, or less than 2 mm, or less than 1 mm.

The external device, for example sheet, may be flexible. The device, for example sheet, may be elastic. The external device, for example sheet, may be curved.

Where the external device is headgear, the device, for example sheet, may be shaped to conform to the temporal, parietal, frontal or occipital bones of a cranium, or combinations and variations thereof. The external device, for example sheet, may be shaped to conform to the left or right sides of the cranium.

The external device, for example sheet, may be manufactured as a pre-shaped sheet. It may be pre-shaped to conform to the surface contours of the internal or external anatomy of the wearer. That is, the external device, for example sheet, is preferably not a flat sheet that has been curved in a single dimension, but rather a sheet that has been manufactured to substantially conform to the contours of internal or external patient anatomy. For example, the external device, for example sheet, may be substantially dome shaped so as to conform to a respective dome shaped contour of patient anatomy. The external device, for example sheet, and the contour of external wearer anatomy may have a substantially hand and glove relationship.

Where the external device is headgear, it may be based on the form of a conventional protective helmet such as might be worn by cyclists or other sportspeople, for example rugby or Australian Rules Football players or North American Football (Gridiron) players. In this embodiment, a support structure intended to distribute the force of any impact applied to the helmet to the skull bone around the skull defect may be designed using the anatomical data. A preferred method of manufacturing this support structure would be using additive manufacturing, however it could also be manufactured using subtractive techniques, injection moulding and the like.

In one embodiment, one or more elements of a helmet structure, including inner shells for providing optimal anatomical contour, and outer shells for providing protection, or both, may be designed using the anatomical data of the wearer, and manufactured using additive manufacturing techniques to provide optimal fit, comfort and therefore protection to the brain of a patient, for example a craniectomy patient.

In one embodiment, the external device anatomically conforms precisely to the contour of the skull of the intended patient. The contour of the skull is obtained and determined by using data provided from one or more imaging devices.

The external device may, at least in part, be manufactured using additive manufacturing. It will be appreciated that the practice of applying a heated thermoplastic directly to the external anatomy of the patient may result in injury and/or suboptimal precision of the contour, with the added potential for discomfort of the patient and/or suboptimal protection of the anatomy. Advantageously, additive manufacturing provides the ideal method of manufacturing an external device based on the anatomical data provided from the one or more imaging devices.

In one embodiment the external device may be a full body suit or any part thereof. For example, the external device may be a suit or any part thereof manufactured from a suitable elastic material that closely fits the patient's external anatomy. Non-limiting elastic materials include Spandex®, Lycra® and the like.

The suit, or any part thereof, may comprise one or more functional elements as herein disclosed suitably positioned and/or orientated within the suit, or parts thereof, so as to substantially align with specific surface regions of the external anatomy of the patient. Accordingly, optimum and advantageous operation of the functional elements may result.

The one or more functional elements may be in communication with one or more remote devices and functional elements in the bone reconstruction implant. The one or more functional elements may form a network comprising one or more remote devices and the bone reconstruction implant. Remote devices include, but are not limited to, mobile communication devices such as phones, remote computer servers and the like.

The one or more functional elements may be physically connected to one or more functional elements in one or more devices implanted in the wearer, including a bone reconstruction implant.

Further implanted devices include, but are not limited to, sensory devices, neurological devices, cardiovascular devices, orthopaedic devices, contraceptive devices and cosmetic devices.

The connection may be via suitable male and female connectors. The connection may be temporary or permanent.

The one or more functional elements may be in wireless communication with an internally implanted device and the bone reconstruction implant

At least one functional element in the external device may also align with an internal anatomical feature of the patient.

Internal anatomical features of the patient include, but are not limited to, vascular features, physical features, functional features and electrical features or combinations thereof. Vascular features, such as vascular topography, may be determined by, for example, vascular mapping. Electrical features may be determined by, for example, electroencephalography.

By aligns it is meant that the position of the at least one functional element in the external device is such that its interaction with a target internal feature of the patient is substantially maximized.

Examples of functional elements include, but are not limited to, sensors, for example, temperature sensors, biochemical sensors, mechanical sensors, electrical sensors, ultrasonic sensors or optical sensors; control elements, for example, fluid/pressure control elements; elements which modulate the functioning of a tissue, for example, stimulating electrodes, stimulating electromagnets, light sources, ultrasonic emitters; power generation elements; elements which deliver or receive energy. Functional elements may also include any associated components on which the operation of the aforementioned elements depend, for example electronics, tubing, wires, support structures and encasing structures.

The functional elements in the external medical device may be of any three dimensional shape. Preferred three dimensional shapes include, for example, a sphere, a spheroid, a cube, a rectangular prism, a prism, a triangular prism, a cylinder, a cone, a pyramid, or combinations thereof.

The functional elements may provide means for one or more of sensing, communicating, actuating, delivering or receiving information, delivering or receiving energy, or generating energy.

The means for sensing may be one or more of chemical, electrical, physical, optical or magnetic.

The means for stimulating may be one or more of chemical, electrical, physical, optical or magnetic.

The means for actuating may be one or more of pressure, vacuum or physical deformation.

The means for energy generation may be, for example, based on physical movement of the patient or based on other movements.

The functional elements may provide means for monitoring and/or measuring patient specific parameters and/or non patient specific parameters.

Patient specific parameters include, but are not limited to, temperature, pressure, electrophysiological changes or the concentration and/or nature of one or more chemical species.

Non patient specific parameters include, but are not limited to, temperature, pressure, light intensity, electromagnetic radiation, sound, or the concentration and/or nature of one or more chemical species.

The one or more functional elements may provide means for stimulating the wearer.

The one or more functional elements may be in communication with one or more implanted devices. The one or more functional elements may deliver electrical power to, for example, an implanted device.

In one embodiment the functional elements may sense and quantify environmental parameters, for example, light, sound, electromagnetic radiation, thermal radiation or gravitational forces.

In one embodiment the functional elements may comprise GPS and satellite communication systems.

In one embodiment the functional elements may allow remote monitoring and feedback.

The one or more functional elements may be in communication with each other. The one or more functional elements may form or be part of a communication network.

The one or more functional elements may comprise one or more electronic components.

The one or more electronic components may provide means for one or more of sensing, stimulating, communicating, actuating, delivering or receiving information, delivering or receiving energy, or generating energy.

In one embodiment the external medical device may comprise more than one functional element positioned within different regions of the device. For example, the external medical device may be a body suit or parts thereof.

The positioning of the one or more functional elements in the external medical device may be determined using computer software via automated processes.

For example computer imaging data may be obtained from one or more imaging techniques, that is, any technique either singly or in combination that can capture and represent, for example, in digital form, the internal anatomy of the human body (the internal anatomical data) of the patient.

Examples of such techniques include x-ray radiographic, magnetic resonance imaging, medical ultrasonography, medical ultrasound, endoscopy, elastography, tactile imaging, thermography, medical photography, positron emission tomography (PET) and single photon emission computed tomography.

Measurement and recording techniques which are not primarily designed to produce images, such as electroencephalography (EEG), magnetoencephalography (MEG) and electrocardiography (ECG) may also be employed to determine the positioning of the functional elements in the external medical device.

When positioned on a patient's head the external device may be headgear. Examples of headgear include, but are not limited to, a helmet, a cap, and a headband.

In one embodiment the external device may comprise a sheet of, for example, a thermoplastic material. The external device may comprise a thin sheet. The external device may comprise an impact-resistant thermoplastic. The external device may be aesthetically-pleasing.

The external device, for example sheet, may have a thickness between about 0.05 mm and 10 mm, or between about 0.1 mm and about 5 mm, or between about 0.2 mm and about 2 mm. The external device, for example sheet, may have a thickness less than 10 mm, or less than 5 mm, or less than 2 mm, or less than 1 mm.

The external device, for example sheet, may be flexible. The device, for example sheet, may be elastic. The external device, for example sheet, may be curved.

The external device may, at least in part, be manufactured using additive manufacturing. It will be appreciated that the practice of applying a heated thermoplastic directly to the external anatomy of the patient may result in injury and/or suboptimal precision of the contour, with the added potential for discomfort of the patient and/or suboptimal protection of the anatomy. Advantageously, additive manufacturing provides the ideal method of manufacturing an external device based on the anatomical data provided from the one or more imaging devices.

In one embodiment the external device may be a full body suit or parts thereof. For example, the external device may be a suit or parts thereof manufactured from a suitable elastic material that closely fits the patient's external anatomy. Non-limiting elastic materials include Spandex®, Lycra® and the like.

The suit, or parts thereof, may comprise one or more functional elements as herein disclosed suitably positioned and/or orientated within the suit, or parts thereof, so as to substantially align with functional elements in one or more internal devices, including a bone reconstruction implant. Accordingly, optimum and advantageous operation of the functional elements may result.

The one or more functional elements may be in communication with one or more remote devices. The one or more functional elements may form a network comprising one or more remote devices. Remote devices include, but are not limited to, mobile communication devices such as phones, remote computer servers and the like.

The one or more functional elements may be physically connected to one or more functional elements in a device implanted in the patient.

The connection may be via suitable male and female connectors. The connection may be temporary or permanent.

The one or more functional elements may be in wireless communication with an internally implanted device.

Throughout this specification the term ‘aligns’, in the sense of alignment of functional elements in the bone reconstruction implant with functional elements in an implanted device or external device, may mean that the position of the at least one functional element in the bone reconstruction implant is such that its interaction with a functional element in the implanted device or functional element in the external device is substantially maximized

Alternatively or additionally, throughout this specification the term ‘aligns’, in the sense of alignment of functional elements in the bone reconstruction implant with functional elements in an implanted device or external device, may be mean that at least one functional element in the bone reconstruction implant and at least one functional element in the implanted device or external device are within proximity such that energy transfer can occur.

Alternatively or additionally, throughout this specification the term ‘aligns’, in the sense of alignment of functional elements in the bone reconstruction implant with functional elements in the implanted device or external device, may be mean that at least one functional element in the bone reconstruction implant and at least one functional element in the implanted device or external device overlap in at least one respective surface by at least 10%, or at least 20%, or at least 30%, or at least 40%, or at least 50%, or at least 60%, or at least 70%, or at least 80%, or at least 90%, or substantially 100%.

Alternatively or additionally, throughout this specification the term ‘aligns’, in the sense of alignment of functional elements in the bone reconstruction implant with functional elements in the implanted device or external device, may mean that at least one functional element in the bone reconstruction implant and at least one functional element in the implanted device or external device are positioned relative to one another so that the angle between a longitudinal axis through the functional element in the bone reconstruction device and a longitudinal axis through the functional element in the implanted device or external device is less than 90°, or less than 80°, or less than 70°, or less than 60°, or less than 50°, or less than 40°, or less than 30°, or less than 20°, or less than 10°, or substantially 0°. When the angle is substantially 0°, then the functional element in the bone reconstruction implant and the functional element in the implanted device or external device are substantially parallel with each other.

Alternatively or additionally, throughout this specification the term ‘aligns’, in the sense of alignment of functional elements in the bone reconstruction implant with functional elements in the implanted device or external device, may mean that the centre of at least one functional element in the bone reconstruction implant and the centre of at least one functional element in the implanted device or external device are offset from each other by no more than 10%, or no more than 20%, or no more than 30%, or no more than 40%, or no more than 50%, or no more than 60%, or no more than 70%, or no more than 80%, or no more than 90%, or no more than 100%, relative to the largest dimension of the functional element in the bone reconstruction implant.

The herein disclosed bone reconstruction implant, methods or systems may comprise any one or more of the herein disclosed embodiments for all aspects of the present disclosure. For example, any of the herein disclosed systems may include any one or more of the herein disclosed bone reconstruction implants, and one or more of the herein disclosed other internal implants or attachments and/or any one or more of the herein disclosed external devices in any combination.

### BRIEF DESCRIPTION OF THE DRAWINGS

FIG. 1 illustrates an implant according to an embodiment of the present disclosure.

FIG. 2 illustrates another implant according to an embodiment of the present disclosure.

FIG. 3 illustrates another implant according to an embodiment of the present disclosure.

FIG. 4 illustrates another implant according to an embodiment of the present disclosure.

FIG. 5 illustrates another implant according to an embodiment of the present disclosure.

FIG. 6 illustrates another implant according to an embodiment of the present disclosure.

FIG. 7 illustrates another implant according to an embodiment of the present disclosure.

FIG. 8 illustrates another implant according to an embodiment of the present disclosure.

FIG. 9 illustrates another implant according to an embodiment of the present disclosure.

FIG. 10 illustrates a system according to an embodiment of the present disclosure.

FIG. 11 illustrates a system according to an embodiment of the present disclosure.

FIG. 12 illustrates a system according to an embodiment of the present disclosure.

FIG. 13 illustrates a system according to an embodiment of the present disclosure.

### DESCRIPTION OF EXEMPLARY EMBODIMENTS

Throughout this specification, use of the terms “comprises” or “comprising” or grammatical variations thereon shall be taken to specify the presence of stated features, integers, steps or components but does not preclude the presence or addition of one or more other features, integers, steps, components or groups thereof not specifically mentioned.

Before the present devices and/or methods and/or systems are disclosed and described, it is to be understood that unless otherwise indicated this disclosure is not limited to specific devices, components, systems, designs, methods, or the like, as such may vary, unless otherwise specified. It is also to be understood that the terminology used herein is for the purpose of describing particular embodiments only and is not intended to be limiting.

It must also be noted that, as used in the specification and the appended claims, the singular forms ‘a’, ‘an’ and ‘the’ include plural referents unless otherwise specified. Thus, for example, reference to ‘an implant’ may include more than one implants, and the like.

Disclosed herein are advantageous implants, methods and systems for delivering health care. The implants, methods and systems may be based on patient specific information or patient specific anatomy. Accordingly, a patient specific implant contoured to a patient's bony anatomy may be provided.

In some embodiments, the bone reconstruction implant of the present disclosure contains one or more permanently-housed functional elements, the elements being encapsulated within the substance of the implant and wherein the elements cannot be removed without disrupting the physical integrity of the implant. In other embodiments, the one or more elements are housed within one or more voids incorporated within the implant.

The one or more voids may be incorporated in the implant in a variety of ways.

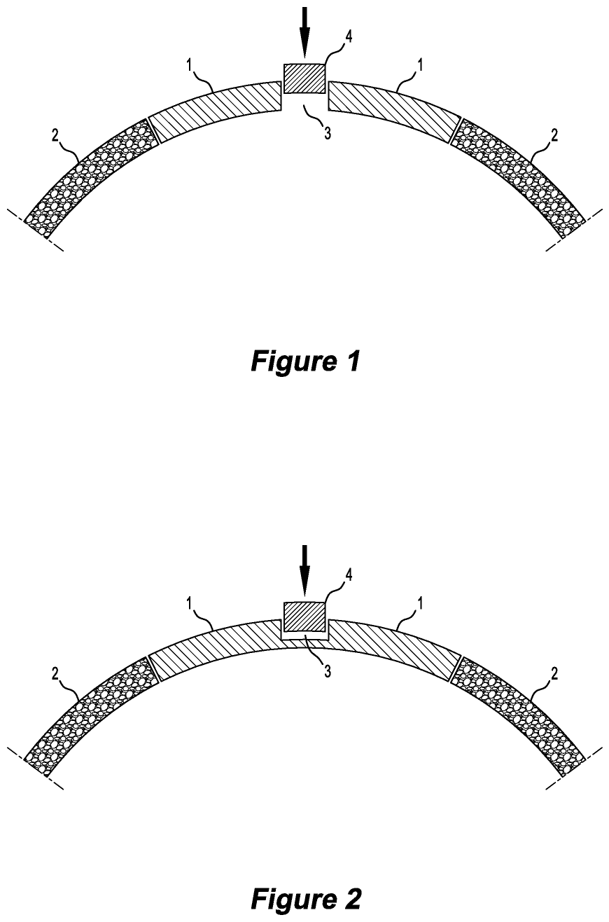

In one embodiment the one or more voids may span the entire thickness of one or more dimensions of the implant. FIG. 1 illustrates a cross section of such a bone reconstruction implant (1) implanted in bone (2). The implant contains void (3) which houses functional element (4). The void spans the entire thickness of the vertical dimension of the implant.

In other embodiments the one or more voids may span a partial thickness of the implant. FIG. 2 illustrates a cross section of bone reconstruction implant (1) implanted in bone (2). The implant contains void (3) which houses functional element (4). The void spans only a partial thickness of the vertical dimension of the implant.

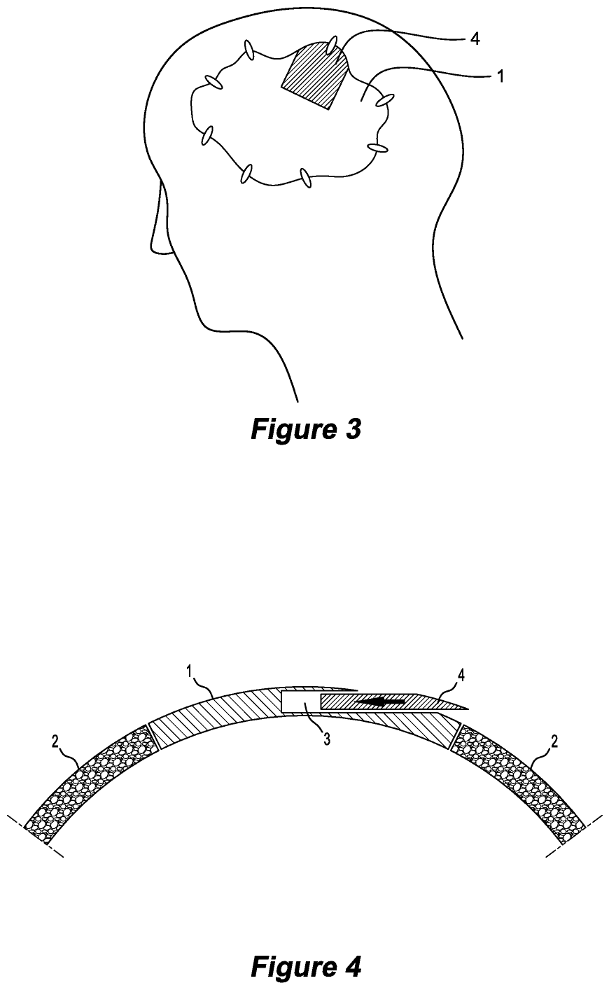

In one embodiment, wherein one or more voids constitute part of the outer edge of the implant, the one or more functional elements intended to occupy the one or more voids may be designed and fabricated such that their dimensions and shape are consistent with the anatomy of the bony defect into which the implant is placed. This embodiment is illustrated in FIG. 3 where implant (1) contains functional element (4). In this embodiment, both the bone reconstruction implant and the one or more functional elements are contoured so that their shapes are ‘patient specific’.

FIG. 4 illustrates a cross section of a bone reconstruction implant (1) implanted in bone (2). The implant contains void (3) which houses functional element (4). In this embodiment the functional element is positioned in the void such that a single surface is exposed.

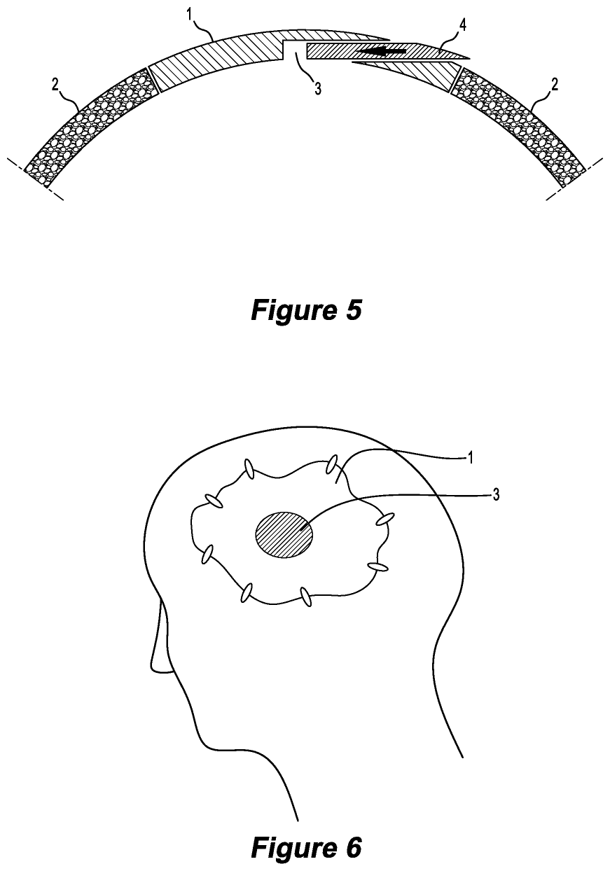

FIG. 5 illustrates a cross section of a bone reconstruction implant (1) implanted in bone (2). The implant contains void (3) which houses functional element (4). In this embodiment the functional element is positioned in the void such that two surfaces are exposed.

The one or more voids may constitute a large area or volume of the bone reconstruction implant. The one or more voids may constitute a small area or volume of the implant. The one or more voids may constitute part of the outer edge of the implant. The one or more voids may be contained entirely within the perimeter of the implant. This embodiment is illustrated in FIG. 6, where implant (1) contains void (3). The one or more voids may be of any shape. The one or more voids may be substantially circular in shape. The one or more voids may be substantially square in shape. The skilled artisan will appreciate that the one or more voids may be designed and fabricated to conform to any shape, and in a variety of, or combinations of shapes.

The implants may be curved in shape. The implants may be non-curved in shape.

One or more of the one or more voids may be designed such that a functional element may be inserted at an angle of about 90 degrees to the implant at the point of insertion (as illustrated in FIG. 1). Alternatively, the angle of insertion may be substantially parallel to the implant at the point of insertion (as illustrated in FIG. 5). In further embodiments, one or more functional elements may be inserted into one or more voids at a variety of insertion angles.

In one embodiment of the presently disclosed implant, wherein one or more of the one or more functional elements are housed within a void, the one or more functional elements may be intended to be permanently housed, for example by means of permanent fixation. Examples of such permanent fixation include by use of a biocompatible glue, or subsequent to bony ingrowth into the outer margins of the one or more functional elements. These examples are not intended to be limiting, and the skilled artisan will appreciate that other methods of permanent fixation, not herein described, may be utilised.

In one embodiment of the implant, wherein one or more of the one or more functional elements are contained within a void, the one or more functional elements may be removable. The one or more functional elements may be secured in place by using standard surgical fixation hardware including but not limited to screws, plates, rivets and the like. In another embodiment, the one or more functional elements may be secured by a screw mechanism. That is, a void may contain a receiving thread, into which the outer threaded surface of a functional element may be screwed to achieve fixation of the functional element. In another embodiment, a functional element may be secured by a keyed sliding mechanism, for example there may be receiving channels in a void, into which physical extensions of the outer surface of a functional element are designed to fit. The skilled artisan will appreciate that numerous securing or interlocking mechanisms may be utilised to ensure firm attachment of one or more functional elements into a void, and the examples given are not intended to be limiting.

It may be advantageous for the one or more functional elements to have a predictable alignment to an adjacent body structure, for example a tissue, and therefore the securing mechanism may be “keyed”. That is, the securing mechanism ensures a functional element being inserted into a void is required to be inserted in a manner that optimises the proximity of functional elements to a bodily structure or tissue. An example may be given in the case of a cranial implant containing a void, into which a housing containing electrodes on its outer surface is inserted at an angle approximately parallel to a region of the implant. To ensure the electrodes face the underlying brain correctly, the outer surface of the housing and the inner surface of the void may be “keyed” to guarantee correct orientation of the housing and therefore electrodes with respect to the brain surface.

In one embodiment, wherein the bone-reconstruction implant contains both permanent and removable functional elements, the permanent and removable functional elements may operate independently. In another embodiment, the permanent and removable functional elements perform a combined function.

In one embodiment, the permanent and removable functional elements may be physically connected, thereby facilitating the transfer of energy, information, or other functionally-relevant properties from one functional element to another.

In an embodiment of the bone-reconstruction implant containing one or more voids within which one or more functional elements may be housed, it will be appreciated that, where appropriate, it may be advantageous for the voids to be of a shape and dimensions that are consistent from one implant to the next. Therefore, a functional element of standard shape and size may be designed and manufactured to fit into the one or more voids. Thus, by combining the methods of mass manufacture and patient-specific implant design, an optimal combination of optimal anatomic fit and cost of manufacture can be achieved. Alternatively, as clinical needs dictate, the shape of the one or more voids, and therefore the shape of the one or more functional elements, may be designed and manufactured on an individual basis. Thus, the bone-replacing implant may contain one or more patient specific voids, into which one or more patient-specific functional elements may be inserted. Alternatively, the implant may contain one or more voids of a consistent size and shape (i.e. “standardised voids”), into which one or more functional elements of consistent size and shape (i.e. “off the shelf” functional elements) may be inserted. Alternatively, the implant may contain a combination of patient specific voids and functional elements, and standardised voids and off the shelf functional elements.

It will be appreciated that a combination of anatomical data obtained using one or more imaging devices and additive manufacturing provides an advantageous method by which patient specific bone-reconstruction implants may be fabricated. Furthermore, it will be appreciated that a combination of anatomical data obtained using one or more imaging devices and additive manufacturing provides an advantageous method by which a patient-specific, bone-reconstruction implant may be fabricated, within which one or more or a combination of patient-specific voids or standardised voids may be incorporated.

The one or more functional elements may comprise one or more functional or structural components. The structural components may comprise housings or casings, fixation devices (for example, screws, rivets and the like) or the aforementioned components of a system intended to secure a functional element to a void.

The one or more structural components may be manufactured from a variety of materials, including but not limited to soft or hard plastics/polymers, or materials with a continuum of physical properties that would render these materials as being soft, hard, or anywhere in between, metals (such as titanium and other metals or metal alloys that are amenable to implantation), ceramics, or composites of these materials. Those skilled in the art will appreciate that other biocompatible materials may be used, and the examples given should not be taken to be limiting.

The structural components may be manufactured using additive manufacturing techniques, or by conventional techniques such as subtractive manufacturing (for example, CNC machining or multi-axis routing), injection moulding and so on.

The functional elements may be contained entirely within the structural components; that is to say that the structural components may completely encapsulate the functional elements. Alternatively, the functional elements may form an integral part of the structural components. For example, a functional element may comprise structural components being a polymer casing within which functional components, being electronic components, are contained, whilst the exterior of the casing contains integrated electrodes, connected to the electronic components, for delivering stimulus pulses to a subjacent tissue.

The functional components may be partly or entirely separable from the structural components, or the functional components may be only partially separable. The functional components and structural components may form a single construct, thereby rendering the functional components entirely non-separable from the structural components. For example, a functional device may comprise electronic components encapsulated in a polymer, wherein the polymer is moulded around the electronic components.

The functional components may comprise one or more of, circuit boards, electronic components (for example processors), energy storage devices, energy generation devices and so on. In one embodiment, the functional components may comprise one or more devices intended to diagnose, treat or otherwise modify the activity, functioning or physical properties of a biological tissue, biological fluid or physiological system. Such devices may include, but are not limited to, electrodes (for example electrodes for recording electrical or electrophysiological signals, or for delivering or receiving electrical charge), electromagnets, optical emitters or receivers, pressure sensors, chemical sensors, mechanical, electromechanical or microelectromechanical devices including ultrasonic emitters or receivers, fluid or gas pumps and valves, tubes (for example for fluid removal or administration) and so on.

The tissue may be human tissue or animal tissue. The tissue may be in close proximity to the one or more devices. The tissue may physically contact the one or more devices. The tissue may be remote from the one or more devices. The fluid may be cerebrospinal fluid. The fluid may be blood. The fluid may be other bodily fluids.

In one embodiment, where the tissue is brain, the tissue may be cerebral cortex. The tissue may be white matter. The tissue may comprise the anatomical structures of the basal ganglia. The tissue may comprise the anatomical structures of the brainstem. The tissue may comprise the cerebellum. The tissue may be white matter. The tissue may be grey matter. The tissue may be a combination of grey and white matter.

In one embodiment, the functional components comprise one or more devices intended to communicate with a remote device. In another embodiment, the one or more devices comprise devices intended to receive power from, or deliver power to, a remote device. Examples of remote devices may include a mobile telecommunications device such as a mobile phone or other device employing radiofrequency communications technologies. Another example of a remote device may be a hand-held instrument placed over the implant, wherein the instrument sends a signal to the implant such that a device in the implant receives power and data from the instrument. The implant may then return a signal back to the instrument, and the instrument may process that signal.

The one or more functional components may comprise one or more devices intended to generate power, for example the one or more devices may comprise kinetic energy harvesting technology or biological fuel-cell technology, or other energy-generation technologies amenable to implantation.

In one embodiment, it may be advantageous to ensure that the locations of the functional components comprising one or more devices intended to diagnose, treat or otherwise modify the activity, functioning or physical properties of a biological tissue matches the anatomy and/or functional topography of the tissue. For example, in one embodiment, wherein the tissue is brain, it may be advantageous that the locations of the one or more devices be aligned with the gyri within the cortical area over which the bone-replacing implant will be placed.

In yet another embodiment, it may be advantageous that the locations of the one or more functional elements be aligned with functionally-specific regions of, for example, the brain. In this embodiment, functionally-specific regions of, for example, the brain may be identified by the use of imaging devices or techniques including, but not limited to, functional Magnetic Resonance Imaging (fMRI), Magnetoencephalography (MEG) and Positron Emission Tomography (PET). Alternatively, the locations of functionally-specific regions of, for example, the brain may be identified through the prior performance of, for example, cognitive and/or motor tasks during electrophysiological recording with subdural or epidural electrode arrays.

In one embodiment, wherein the bone-reconstruction implant contains both permanent and removable functional elements, the permanent and removable functional elements may operate independently. In another embodiment, the permanent and removable functional elements perform a combined function.

Moreover, it may be advantageous that the locations of the one or more functional elements be designed to avoid vascular structures. Alternatively, it may be advantageous for some of the one or more functional elements to deliberately overlay vascular structures, whilst some do not. It should thus be recognised that, given the anatomy of the tissue will vary from one person to another, that the locations of the one or more functional elements is patient specific.

In one embodiment, wherein the locations of the one or more functional elements is patient specific, the intended locations may predetermined using anatomical data, itself obtained using imaging devices, prior to manufacture of the one or more functional elements. Imaging devices may include magnetic resonance imaging scanners, computed tomography scanners, medical ultrasound systems, one or more lasers, one or more cameras, and other devices known to those skilled in the art to enable the imaging of external or internal anatomy.

In one embodiment, the one or more functional elements constitutes a “brain machine interface”, wherein the one or more functional elements facilitates communication between the implant recipient and external technology, for example robotic assistive devices or computers.

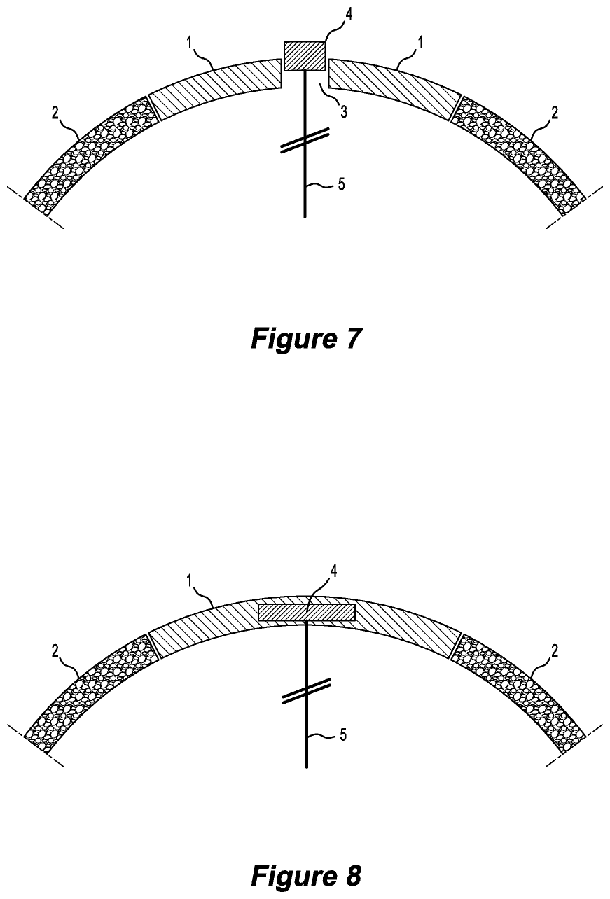

In one embodiment, the one or more functional elements are connected to one or more attachments that extend beyond the borders of the bone-reconstruction implant. For example, FIG. 7 illustrates a cross section of a bone reconstruction implant (1) implanted in bone (2). The implant contains void (3) which houses functional element (4). Attachment (5) is attached to the functional element (4). FIG. 8 illustrates a cross section of a bone reconstruction implant (1) implanted in bone (2). The implant contains void (3) which houses functional element (4). Attachment (5) is attached to the functional element (4).

In one embodiment the one or more attachments may be permanently connected to the one or more functional elements or the one or more attachments may be detachable. The one or more attachments may be dissolvable or resorbable. One or more of the one or more attachments may penetrate the tissue. One or more of the one or more attachments may rest on the surface of the tissue.

The one or more attachments may comprise one or more devices intended to diagnose, treat or otherwise modify the activity, functioning or physical properties of a biological tissue, biological fluid or physiological system. Such devices may include, but are not limited to, electrodes (for example electrodes for recording electrical or electrophysiological signals, or for delivering or receiving electrical charge), electromagnets, optical emitters or receivers, pressure sensors, chemical sensors, mechanical, electromechanical or microelectromechanical devices including ultrasonic emitters or receivers, fluid or gas pumps and valves, tubes (for example for fluid removal or administration) and so on. The one or more attachments may comprise one or more devices intended to communicate with a remote device. The one or more attachments may comprise one or more devices intended to receive power from, or deliver power to, a remote device. The one or more attachments may comprise one or more devices intended to generate power.

The biological tissue may be human tissue or animal tissue. The tissue may be in close proximity to the one or more devices. The tissue may physically contact the one or more devices, for example in the case of electrodes, whereby the electrodes are positioned on a tissue-contacting surface of the attachment. The tissue may be remote from the one or more devices. The fluid may be cerebrospinal fluid. The fluid may be blood. The fluid may be other bodily fluids.

The form of the one or more attachments may comprise a structure generally regarded as a lead or cable, or a structure generally regarded as a sheet or layer, or a combination thereof. The lead or cable may be thin, for example the lead or cable may be less than 1 mm in diameter, or the lead or cable may be between 1 mm in diameter and 3 mm in diameter. The lead or cable may be larger than 3 mm in diameter. The sheet or layer may comprise one or more sub-layers, that is the sheet may be laminar in structure. The sheet or layer may be connected to the one or more devices in the bone replacing implant by means of a cable or wire. The sheet or layer may be connected by means of a highly flexible, narrower sheet, such as would be generally regarded as a “ribbon cable”. The one or more attachments may comprise combinations of sheets and cables.

The lead, cable, sheet or layer may comprise, for example, a thermoplastic material. The lead, cable, sheet or layer may be made of silicone, polytetrafluoroethylene, polyurethane or other suitable biocompatible materials.

The lead, cable, sheet or layer may be flexible. The lead, cable, sheet or layer may be elastic. The lead, cable, sheet or layer may be biodegradable. The lead, cable, sheet or layer may be biostable. The lead, cable, sheet or layer may be curved.

In one embodiment the lead, cable, sheet or layer may be shaped to conform to part of a patient's internal anatomy.

The lead, cable, sheet or layer may be manufactured as a pre-shaped form. They may be pre-shaped to conform to the surface contours of internal patient anatomy. For example, the sheet may not be a flat sheet that has been curved in a single dimension, but rather a sheet that has been manufactured to substantially conform to the contours of patient anatomy. For example, the sheet may be substantially dome shaped so as to conform to a respective dome shaped contour of internal patient anatomy. The sheet and the contour of patient internal anatomy may have a substantially hand and glove relationship.

In one embodiment wherein one or more attachments comprises one or more devices, it is advantageous to ensure that the locations of the one or more devices matches the anatomy and/or functional topography of the tissue. It should thus be recognised that, given the anatomy of the tissue will vary from one person to another, that the locations of the one or more devices is patient specific. When the locations of the one or more devices is patient specific, the intended locations may predetermined using anatomical data, itself obtained using imaging devices, prior to manufacture of the one or more attachments. Imaging devices may include magnetic resonance imaging scanners, computed tomography scanners, medical ultrasound systems, one or more lasers, one or more cameras, and other devices known to those skilled in the art to enable the imaging of external or internal anatomy.

In yet another embodiment, it may be advantageous that the locations of the one or more devices be aligned with functionally-specific regions of, for example, the brain. In this embodiment, functionally-specific regions of, for example, the brain may be identified by the use of imaging devices or techniques including, but not limited to, functional Magnetic Resonance Imaging (fMRI), Magnetoencephalography (MEG) and Positron Emission Tomography (PET). Alternatively, the locations of functionally-specific regions of, for example, the brain may be identified through the prior performance of, for example, cognitive and/or motor tasks during electrophysiological recording with subdural or epidural electrode arrays.

In certain circumstances, the shape or anatomy of a tissue or bodily structure that is in ordinarily in close proximity to a bony structure may alter over time. Examples of such circumstances include, but are not limited to, the atrophy of brain tissue, or encephalomalacia that may occur after brain trauma, stroke, or a neurosurgical procedure. In such cases, the distance from the brain surface to the inner surface of the skull, or the skull defect introduced by the performing of a decompressive craniectomy, may alter over time.

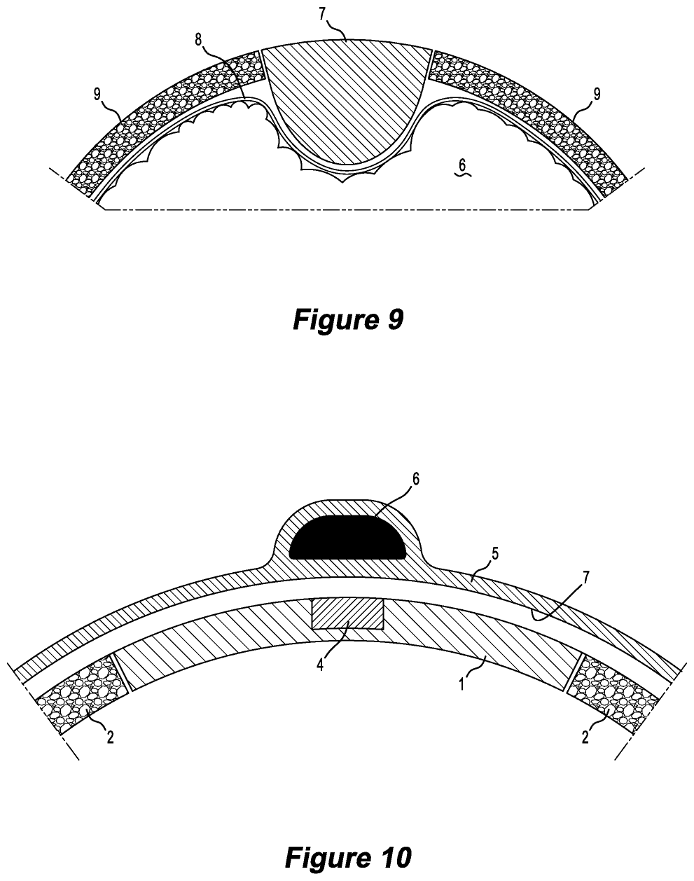

It will be appreciated that it may be advantageous that the one or more devices or one or more attachments remain in close proximity to the tissue, in order to maintain their ability to perform their intended function. Thus, in one embodiment of the device described herein, the one or more attachments are highly flexible, or any leads or ribbon cables connecting the one or more attachments are extendable or possess sufficient excess length to permit extension without stretching. In another embodiment, in which the one or more attachments comprise a sheet, the contour of the sheet may be dynamically altered to fill any void that exists or develops between the sheet and the tissue. In still another embodiment, the contour or anatomy of the bone reconstruction implant does not replicate the original anatomy of the bone it is intended to replace. In this embodiment, the contour of the bone-reconstruction implant is designed, prior to manufacture, to ensure maximal contact with an adjacent or surrounding tissue, for example, wherein the tissue has moved from its usual anatomical position. An example of this embodiment is shown in FIG. 9 where implant (7) affords maximum contact with adjacent skull bone (9), dura (8) and brain (6).

In one embodiment of the bone-reconstruction implant containing a void for one or more devices, the one or more devices may comprise one or more reservoirs. The one or more reservoirs may have walls that are elastic or deformable, or the walls may be rigid. The one or more reservoirs may be empty at the time of implantation. The one or more reservoirs may contain a fluid at the time of implantation. The one or more reservoirs may be under vacuum at the time of implantation. The fluid may be sterile. The fluid may be a therapeutic substance. The fluid may be a vasoactive substance. The fluid may be any substance that is known to influence the functioning of biological tissues, biological fluids or physiological systems. The fluid may be a CSF-mimicking fluid. The fluid may be a blood-mimicking fluid. The one or more reservoirs may be connected to one more devices intended to allow the movement of fluid from one location to another. For example, in one embodiment the reservoir may be connected to a pump and tube, wherein the tube provides a fluid path from the cerebral ventricles to the reservoir and the pump serves as a means with which to move the fluid from the cerebral ventricles to the reservoir. In another embodiment, the reservoir, may have deformable walls made of a polymer or otherwise flexible/elastic substance, and is able to be penetrated by a needle for the purposes of withdrawing cerebrospinal fluid. In yet another embodiment, the combination of one or more reservoirs, one or more valves, one or more pressure sensors, one or more tubes, one or more pumps and one or more other devices constitutes a device for the diversion of cerebrospinal fluid from the cerebral ventricles to an extracranial location, for example the atrium of the heart or the peritoneal cavity.

FIG. 10 illustrates, in cross section, a system comprising a bone reconstruction implant and an external device. Bone reconstruction implant (1) implanted in bone (2) contains functional element (4) which is in electronic communication with functional element (6) in external device (5). The external device may be a wearable device and its surface (7) may be customized so that its contours match those of external patient anatomy.

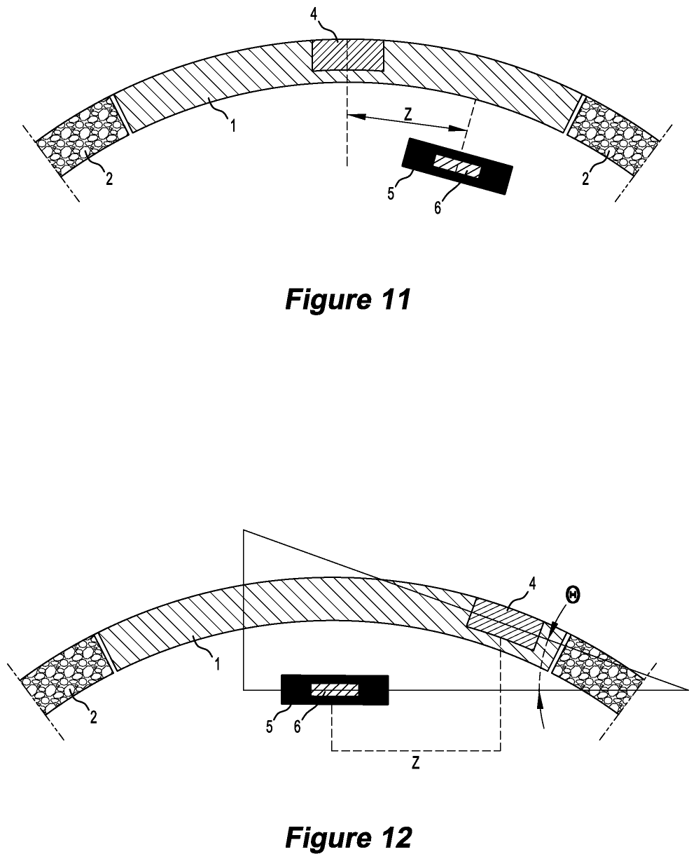

FIG. 11 illustrates, in cross section, a system in which a functional element (6) in implanted device (5) is misaligned with functional element (4) in bone reconstruction implant (1). The centre of the functional element in the implanted device is offset from the centre of the functional element in the bone reconstruction implant by a distance of ‘Z’. Misalignment may occur in any direction. The functional elements are misaligned so that the overlap in at least one respective surface is zero. The functional element in the bone reconstruction implant and the functional element in the implanted device are also misaligned as the centre of the functional element in the bone reconstruction implant and the centre of the functional element in the implanted device are offset from each other (distance ‘Z’) by more than 100%, relative to the largest dimension of the functional element in the bone reconstruction device.

FIG. 12 illustrates a system in which a functional element (6) in implanted device (5) is angularly misaligned with functional element (4) in bone reconstruction implant (1). The centre of the functional element in the implanted device is offset from the centre of the functional element in the bone reconstruction implant by a distance of ‘Z’ and the direction of a longitudinal axis through functional element (6) is offset from the direction through a longitudinal axis through functional element (4) by an angle theta. Misalignment may occur in any direction.

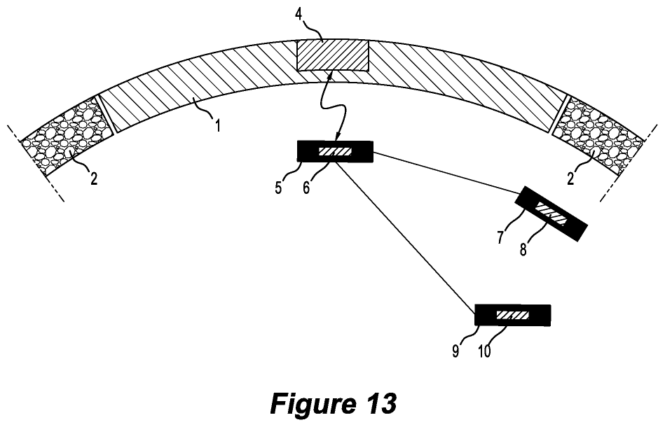

FIG. 13 illustrates a system comprising bone reconstruction implant (1) implanted in bone (2) and containing functional element (4) and implanted device (5) containing functional element (6). The implanted device (5) is implanted in the patient's body. The implanted device (5) is physically connected or wirelessly connected with implanted devices (7) and (9) which respectively contain functional elements (8) and (10). The implanted devices may form part of a single, larger device. Functional elements (4) and (6) facilitate the transfer of energy between the two as denoted by the double arrow. The energy transferred between functional elements (4) and (6) may be for the purposes of providing power, transferring information or other functions as appropriate to the operation of the device. The alignment between functional elements (4) and (6) is optimised to ensure maximum efficiency of energy transfer between the two.

In an exemplary embodiment the present disclosure provides a bone reconstruction implant, said implant comprising one or more functional elements, said implant being contoured in one or more surface regions, said contouring being based on patient specific computer imaging data, so that said one or more surface regions match or substantially match one or more surface contours of a bony defect in said patient and said implant being contoured in one or more further surface regions, said contouring being based on patient specific computer imaging data, so that said one or more further surface regions match or substantially match one or more surface contours of a patient's internal anatomy.

In an exemplary embodiment the present disclosure provides a bone reconstruction implant, said implant comprising one or more functional elements, said implant being contoured in one or more surface regions, said contouring being based on patient specific computer imaging data, so that said one or more surface regions match or substantially match one or more surface contours of a bony defect in said patient and said implant being contoured in one or more further surface regions, said contouring being based on patient specific computer imaging data, so that said one or more further surface regions match or substantially match one or more surface contours of a patient's internal anatomy, said implant comprising one or more voids within which the one or more functional elements are located.

In an exemplary embodiment the present disclosure provides a bone reconstruction implant, said implant comprising one or more functional elements, said implant being contoured in one or more surface regions, said contouring being based on patient specific computer imaging data, so that said one or more surface regions match or substantially match one or more surface contours of a bony defect in said patient and said implant being contoured in one or more further surface regions, said contouring being based on patient specific computer imaging data, so that said one or more further surface regions match or substantially match one or more surface contours of a patient's internal anatomy, said implant comprising one or more voids within which the one or more functional elements are located, said one or more voids spanning the full thickness of at least one dimension of said implant.

In an exemplary embodiment the present disclosure provides a bone reconstruction implant, said implant comprising one or more functional elements, said implant being contoured in one or more surface regions, said contouring being based on patient specific computer imaging data, so that said one or more surface regions match or substantially match one or more surface contours of a bony defect in said patient; said implant being contoured in one or more further surface regions, said contouring being based on patient specific computer imaging data, so that said one or more further surface regions match or substantially match one or more surface contours of a patient's internal anatomy, said implant comprising one or more voids within which the one or more functional elements are located, said one or more voids spanning a partial thickness of at least one dimension of said implant.

In an exemplary embodiment the present disclosure provides a bone reconstruction implant, said implant comprising one or more functional elements, said implant being contoured in one or more surface regions, said contouring being based on patient specific computer imaging data, so that said one or more surface regions match or substantially match one or more surface contours of a bony defect in said patient and said one or more functional elements being contoured in one or more surface regions, said contouring being based on patient specific computer imaging data, so that said one or more surface regions match or substantially match one or more surface contours of a patient's internal anatomy.

In an exemplary embodiment the present disclosure provides a bone reconstruction implant, said implant comprising one or more functional elements, said implant being contoured in one or more surface regions, said contouring being based on patient specific computer imaging data, so that said one or more surface regions match or substantially match one or more surface contours of a bony defect in said patient and said one or more functional elements being contoured in one or more surface regions, said contouring being based on patient specific computer imaging data, so that said one or more surface regions match or substantially match one or more surface contours of a patient's internal anatomy, said implant comprising one or more voids within which the one or more functional elements are located.

In an exemplary embodiment the present disclosure provides a system for monitoring the health of a patient and/or modulating the health of a patient, said system comprising:
(a) at least one bone reconstruction implant said implant being implanted to correct a bony defect in said patient, said implant being contoured in one or more surface regions, said contouring being based on patient specific computer imaging data, so that said one or more surface regions match or substantially match one or more surface contours of a bony defect in said patient; and
(b) at least one other device internal and/or external to said patient's anatomy;
   said at least one other device comprising one or more functional elements;
   wherein at least one functional element in the bone reconstruction implant and at least one functional element in the at least one other device are in electronic communication.

In an exemplary embodiment the present disclosure provides a system for monitoring the health of a patient and/or modulating the health of a patient, said system comprising:
(a) at least one bone reconstruction implant said implant being implanted to correct a bony defect in said patient, said implant being contoured in one or more surface regions, said contouring being based on patient specific computer imaging data, so that said one or more surface regions match or substantially match one or more surface contours of a bony defect in said patient; and
(b) at least one external device positioned on said patient's external anatomy; said at least one external device comprising one or more functional elements;
   wherein at least one functional element in the bone reconstruction implant and at least one functional element in the at least one external device are in electronic communication.

In an exemplary embodiment the present disclosure provides a system for monitoring the health of a patient and/or modulating the health of a patient, said system comprising:
(a) at least one bone reconstruction implant said implant being implanted to correct a bony defect in said patient, said implant being contoured in one or more surface regions, said contouring being based on patient specific computer imaging data, so that said one or more surface regions match or substantially match one or more surface contours of a bony defect in said patient; and
(b) at least one other device internal and/or external to said patient's anatomy; said at least one other device comprising one or more functional elements;
   wherein at least one functional element in the bone reconstruction implant and at least one functional element in the at least one other device are in electronic communication, wherein the at least one functional element in the bone reconstruction implant is substantially aligned with an internal anatomical feature of said patient.

It is contemplated that the devices, methods and systems described herein may find use in a wide range of applications, where implants are required. Thus, where it is desired to deliver healthcare to other parts of the anatomy, the devices, methods and systems of the present disclosure may be used.

It is to be understood that while the present disclosure has been described in conjunction with the specific embodiments thereof, the foregoing description is intended to illustrate and not limit the scope of the disclosure. Other aspects, advantages and modifications will be apparent to those skilled in the art to which the disclosure pertains. Therefore, the above examples are put forth so as to provide those skilled in the art with a complete disclosure and description of how to make and use the disclosed devices, and are not intended to limit the scope of the disclosure.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited.

All documents cited are herein fully incorporated by reference for all jurisdictions in which such incorporation is permitted and to the extent such disclosure is consistent with the description of the present disclosure.

1-58. (canceled)

59. The system for monitoring the health of a patient and/or modulating the health of a patient, said system comprising:
(a) at least one bone reconstruction implant, said implant comprising one or more functional elements, said implant being contoured in one or more surface regions, said contouring being based on patient specific computer imaging data, so that said one or more surface regions match or substantially match one or more surface contours of a bony defect in said patient, said implant being implanted to correct a bony defect in said patient; and
(b) at least one other device internal and/or external to said patient's anatomy, said at least one other device comprising one or more functional elements, wherein at least one functional element in the bone reconstruction implant and at least one functional element in the at least one other device are in communication.

60. A system for monitoring the health of a patient and/or modulating the health of a patient, said system comprising:
(a) at least one bone reconstruction implant, said implant comprising one or more functional elements, said implant being contoured in one or more surface regions, said contouring being based on patient specific computer imaging data, so that said one or more surface regions match or substantially match one or more surface contours of a bony defect in said patient, said implant being implanted to correct a bony defect in said patient; and
(b) at least one external device positioned on said patient's external anatomy, said external device comprising one or more functional elements, wherein the at least one functional element in the bone reconstruction implant and at least one functional element in the external device are in communication.

61. The system according to claim 59, wherein the at least one functional element in the bone reconstruction implant and the at least one functional element in the internal and/or external device are substantially aligned.

62. The system according to claim 59, wherein the at least one functional element in the bone reconstruction implant is substantially aligned with an internal anatomical feature of said patient.

63. The system according to claim 59, wherein the at least one external device comprises one or more surfaces, said one or more surfaces having at least one region contoured to substantially match at least one contour of an external anatomical surface of the patient.

64. The system according to claim 59, wherein at least one of the one or more functional elements in the internal and/or external device and at least one of the one or more functional element in the implanted bone reconstruction implant overlap in at least one respective surface by at least 10%.

65. The system according to claim 59, wherein at least one of the one or more functional elements in the internal and/or external device and at least one of the one or more functional elements in the implanted bone reconstruction implant are positioned relative to one another so that an angle between a longitudinal axis through the functional element in the internal and/or external device and a longitudinal axis through the functional element in the implanted bone reconstruction implant is less than 90°.

66. The system according to claim 59, wherein a center of at least one of the one or more functional elements in the internal and/or external device and a center of at least one of the one or more functional elements in the implanted bone reconstruction implant are offset from each other by no more than 10%, relative to the largest dimension of the functional element in the bone reconstruction implant.

67. The system according to claim 59, wherein the functional element is selected from temperature sensors, biochemical sensors, mechanical sensors, electrical sensors, ultrasonic sensors, optical sensors, fluid/pressure control elements, stimulating electrodes, stimulating electromagnets, light sources, ultrasonic emitters, power generation elements, energy delivery or receiving elements, and combinations thereof.

68. The system according to claim 59, wherein the at least one functional elements are configured for at least one of sensing, communicating, actuating, delivering or receiving information, delivering or receiving energy, generating energy, or modifying the activity, functioning or physical properties of a biological tissue.

69. The system according to claim 59, wherein the at least one functional elements are configured for monitoring and/or measuring patient specific parameters and/or non-patient specific parameters.

70. The system according to claim 59, wherein the patient specific parameters include, temperature, pressure, electrophysiological changes, the concentration and/or nature of one or more chemical species, and combinations thereof.

71. The system according to claim 59, wherein the non-patient specific parameters include temperature, pressure, light intensity, electromagnetic radiation, sound, the concentration and/or nature of one or more chemical species, and combinations thereof.

72. The system according to claim 59, wherein the one or more functional elements sense and quantify environmental parameters, including light, sound, electromagnetic radiation, thermal radiation or gravitational forces, and combinations thereof.

73. The system according to claim 59, wherein the one or more functional elements comprise GPS and satellite communication systems.

74. The system according to claim 59, wherein the one or more functional elements enable remote monitoring and feedback.

75. The system according to claim 59, wherein the one or more functional elements are in communication with one or more functional elements in one or more devices remote from the patient.

76. The system according to claim 75, wherein the one or more devices remote from the patient include at least one of a mobile device or a computer server.

77. The system according to claim 59, wherein at least one functional element in the implanted device is in communication with one or more further functional elements in the implanted device.

78. The system according to claim 59, wherein at least one of the one or more functional elements in the implanted device is in communication with one or more functional elements in one or more further implanted devices in the patient.

79. The system according to claim 59, wherein at least one of the one or more functional elements in the implanted device is removable from the implanted device.

80. The system according to claim 59, wherein at least one functional element in the implanted device is powered by one or more interfaces based on one or more of electromagnetic, optical, ultrasonic and electrical means, or at least one functional element comprises one or more power generation devices comprising energy harvesting technology, biological fuel-cell technology or other energy generation technologies amenable to implantation.
